(19) United States
(12) Patent Application Publication
(10) Pub. No.: US 2023/0255472 A1
(43) Pub. Date: Aug. 17, 2023

(54) EYE MOVEMENT EVALUATION

(71) Applicant: HEADS STOCKHOLM AB, Stockholm (SE)

(72) Inventor: Hans-Peter Kurz, Stockholm (SE)

(21) Appl. No.: 18/016,022

(22) PCT Filed: Jul. 12, 2021
(86) PCT No.: PCT/SE2021/050723

§ 371 Date: Jan. 13, 2023

(30) Foreign Application Priority Data
Jul. 14, 2020 (SE) 2050893-3

(51) Int. Cl. A61B 3/024; A61B 3/00; A61B 3/113

(52) U.S. Cl. CPC A61B 3/024; A61B 3/0091; A61B 3/113

(57) ABSTRACT

The present disclosure relates to a device for providing an eye metric, comprising a display unit (7), producing a visual stimulus (13) to an eye. An eye-tracking unit (9), measures the eye's movements in response to the stimulus, and an analyzing unit, outputting a metric result. The display unit (7) produces a moving stimulus with at least one varying stimulus parameter such as a symbol size, and the eye-tracking unit (9) detects when the eye loses visual contact with the stimulus. The analyzing unit provides a metric result based on the value of the stimulus parameter at the time when loss of visual contact was detected.

### FIELD OF THE INVENTION

The present disclosure relates to a device for providing an eye metric, comprising a display unit, producing a visual stimulus to an eye, an eye-tracking unit, measuring the eye's movements in response to said stimulus, and an analyzing unit, outputting a metric result.

### BACKGROUND OF THE INVENTION

Several eye testing devices are available for testing the eyes of a person. Such tests may be directed to testing the person's eyesight, but also to detect for instance neurological conditions or drug use.

A general problem, with such devices is to enable efficient use thereof, for instance in connection with telemedicine.

### SUMMARY OF THE INVENTION

One object of the present disclosure is therefore to provide a flexible device for testing different eye metrics.

This object is achieved by means of a device as defined in claim 1. More specifically, in a device of the initially mentioned kind, the display unit is configured to produce a moving stimulus with at least one varying stimulus parameter, the eye-tracking unit and the analyzing unit is configured to detect the eye loosing visual contact with the stimulus, and the analyzing unit is configured to provide a metric result based on the value of said stimulus parameter at the time when loss of visual contact was detected.

This gives a substantial flexibility with setting up a test. Different types of stimuli and different ways of varying stimulus parameters allows the testing device to carry out a number of tests simultaneously or sequentially. A single testing device therefore can provide a number of different metrics in an efficient manner, for instance for a telemedicine system.

The varying stimulus parameter may be the contrast between different parts of a moving symbol and/or between the moving symbol and the background.

The symbol may for instance comprise a lighter field and a darker field, and the contrast between the darker field and the lighter field may be gradually decreased.

Alternatively, to or in combination with that effect, pixels of the darker field may be increasingly shuffled.

The average brightness of the symbol may be the same as the background such that the symbol as a whole blends in with the background to some extent.

The stimulus parameter may also be changed by decreasing the size of a moving symbol.

The stimulus parameter may also be changed by changing the velocity, acceleration or turning radius of a moving symbol.

The stimulus may be a symbol moving along a path and repeatedly making jumps in different angles which constitute stimulus parameters. This allows for instance to detect weak or blind sectors in an eye's fovea which may indicate macular degeneration, for example.

Typically, such a jump may be made in a direction deviating from the symbol's direction of movement prior to the jump.

When the tested person loses visual contact with the symbol, an indicator may be provided at the symbol to allow the tested person to regain contact. This allows the system to quickly resume testing. As an alternative, the change of the stimulus parameter may be reversed when the tested person loses of visual contact with the symbol, typically until contact is regained.

The stimulus may be provided with different colours.

The device may include a positioning unit to keep the tested person at a fixed location, the stimulus may include a moving symbol, and the display unit may be configured to produce the moving object at different optical distances from the eye. This allows, for instance, testing of refractive errors.

The display unit may be angled with respect to the location of the tested person such that the change in distance can be increased by producing stimuli at different locations on the display unit.

The display unit may also comprise multiple displays at different distances to the location of the eye.

Typically, the display unit is configured to provide the stimulus in a pattern that is unpredictable to the tested person. In this way, the system more quickly detects the tested person losing track of the stimulus such as a symbol.

The analyzing unit may be configured to provide a metric result based on the value of said stimulus parameter at the time when loss of visual contact was detected and based on a database containing data of a plurality of tested persons having carried out a corresponding test.

A controllable lens may be placed in close proximity to the tested eye and may be configured to change cylindrical and spherical values of that lens. This makes for instance testing of refractive errors more efficient. The control unit may control both the lens and the display device.

### BRIEF DESCRIPTION OF THE DRAWINGS

FIG. 1A schematically illustrates a basic arrangement for carrying out tests.

FIG. 1B illustrates an alternative example where components of the basic setup are included in a VR headset.

FIG. 2 illustrates a first example of a stimulus moving along a path on a screen.

FIG. 3 illustrates one alternative way of varying a stimulus.

FIG. 4 illustrates another alternative way of varying a stimulus.

FIGS. 5-8 illustrate different alternatives for varying the optical distance to a stimulus.

FIG. 9 illustrates an example of a stimulus movement suitable for evaluating age related macular degeneration.

FIG. 10 shows an example where different screens are used for the left and the right eye.

### DETAILED DESCRIPTION OF THE EXEMPLARY EMBODIMENTS

The present disclosure relates in general to devices for providing eye metrics. Such eye metrics may provide an indication of a tested person's eyesight quality, but also other properties such a neurological conditions, drug use, etc.

### Basic Setup

The present disclosure uses an eye tracking functionality that measures the movements of a user's eye or eyes. Usually one eye at a time is tested although in some cases it may be desired to test both eyes simultaneously or alternatingly.

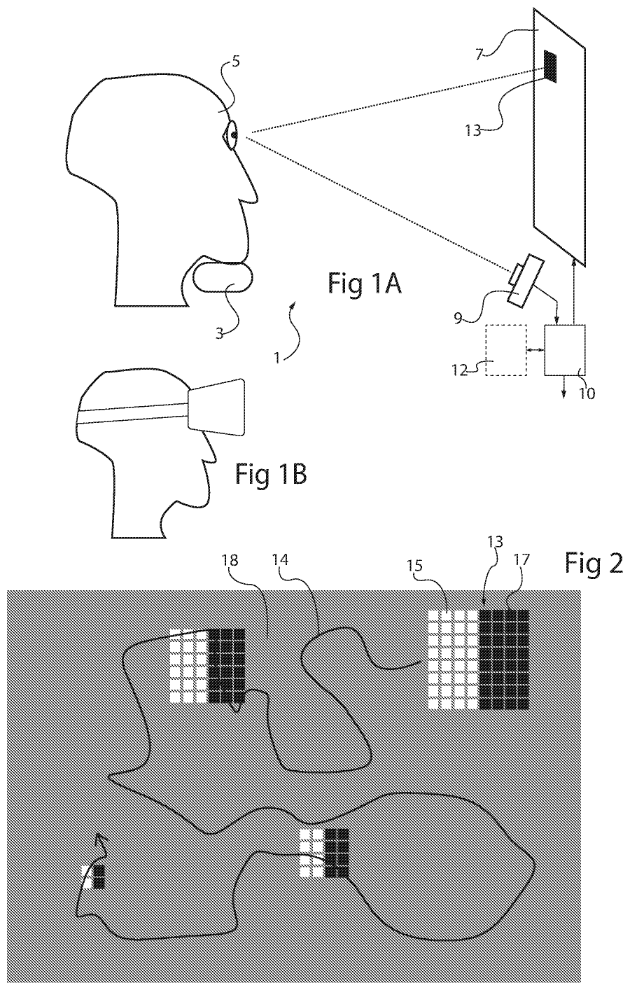

FIG. 1A schematically shows a basic arrangement 1 for carrying out tests. The arrangement may optionally include a head rest 3 where the tested person's head 5 can rest during testing. The test person watches a screen 7 on which various visual stimuli is produced. An eyetracker 9 is used to track the user's eye movements, and an analyzing unit 10 compares the provided stimulus with the eyetracking response to determine a corresponding eye metric and/or other information. The analyzing unit may communicate with a database 12 which contains e.g. corresponding metrics from persons with e.g. known deficiencies etc. in order to provide a more elaborated result as will be discussed.

It should be noted that the arrangement in FIG. 1A is very simplified. A real implementation may include multiple screens, curved screens, wavelength selective mirrors, head motion sensors etc.

The eyetracking may be based on any eyetracking technology, such as so-called bright and/or dark pupil measurements, iris detection, sclera movement observations or glint measurements or a combination thereof, as per se is well known in the art.

The basic device according to the present disclosure thus provides an eye metric by producing, using the display unit or screen 7, a visual stimulus 13 to an eye. The eye-tracking unit 9, measures the eye's movements in response to this stimulus, and the analyzing unit 11, outputs a metric result. The visual stimulus produced moves and has at least one varying stimulus parameter. The eye-tracking unit 9 and/or the analyzing unit is configured to detect the eye loosing visual contact with the stimulus, and-the analyzing unit 10 provides a metric result based on the value of said stimulus parameter at the time when loss of visual contact was detected. That event may indicate that the stimulus no longer appears in or close to the fovea within the eye but further away in the peripheral visual area. This may provide a range of useful information as will be discussed further.

It should be noted that the components of FIG. 1A could be integrated in a virtual reality, VR, headset as schematically illustrated in FIG. 1B. That option implies advantages that will be described in greater detail later.

### Visual Acuity and Contrast Testing

A first use of this concept is determining visual acuity ability, i.e. the tested person's ability to recognize small details with precision. This has traditionally been accomplished by allowing the tested person to read from a so called Snellen-chart, where rows of smaller and smaller letters are shown at some distance.

In the present disclosure, a visual stimulus 13 is shown which moves over the display unit screen 7 as shown in FIG. 2. The stimulus, typically a symbol 13, may move over the screen in a pattern 14 that the tested person cannot anticipate, i.e. a random or random-like fashion, while the symbol e.g. moves quicker or becomes increasingly difficult to distinguish from the background. Thanks to this movement pattern the analyzing unit 11 can determine at which point in time the tested person looses track of the shown symbol as the correlation between the displayed stimuli and the determined eye movements is lost.

There are several ways of varying the difficulty of distinguishing the stimulus from the background. A first option is illustrated in FIG. 2 where the symbol 13 making up the stimulus is rectangular with a white half 15 and a black half 17, side by side. This symbol may be produced against a grey background 18. The symbol 13 as a whole may have the same average greyscale shade as the background, although this is not necessary.

A first option of increasing the difficulty of distinguishing the symbol from the background is to make it smaller in size. As illustrated in FIG. 2, the symbol 13 may shrink e.g. from 8×8 to 6×6 to 4×4 and to 2×2 pixels while moving in an arbitrary pattern over the display surface. This may in principle continue to 2×1 and finally a single pixel, but with many modern displays a single black pixel against a gray background is impossible to distinguish for any eye. The size of the symbol 13 at the time the tested person losses track of its movements, as detected by the eyetracking device, gives an indication of the tested person's acuity.

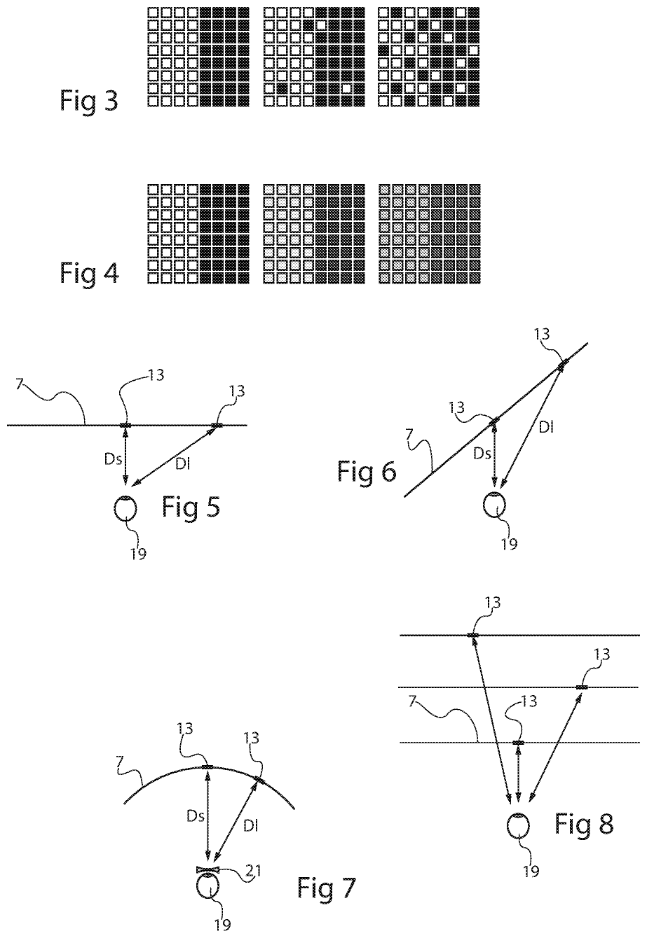

Another way of increasing the difficulty of distinguishing the symbol from the background is illustrated in FIG. 3. In this case, the symbol can optionally retain its size. Initially, the symbol may have a black half and a white half as in the previous example. This gives a strong contrast along the line where the white and black halfs meet. Then, by increasingly shuffling the pixels as seen in the three symbols of FIG. 3, the contrast, both within the symbol and at the periphery of the symbol, becomes decreased as seen by the human eye depending on the eye's and neurological system's acuity. The symbol may finally become chessboard-like.

A third way of increasing the difficulty of distinguishing the symbol from the background is illustrated in FIG. 4. In this case as well, the symbol can optionally retain its size. Initially, as in the previous example, the symbol may have a black half and a white half. In this example, the brightness of the symbol pixels are gradually changed in order to increasingly blend more with the grey background. Thus, the initially black pixels become lighter, and the initially white pixels become darker, optionally until the pixels of the whole symbol have the same brightness of the background and the symbol ceases to exist.

It should be noted that those three ways of altering the symbol can be combined. Additionally, the symbol could optionally rotate, e.g. by changing axis of the black-white transition 90 degrees, for instance.

The symbol need not have a rectangular shape. It would for instance be possible to use circular symbols with alternating angular sectors in black and white, the sector angle of which may decrease over time and/or rotate, for instance.

Another way of varying a stimulus parameter is to change the movement pattern of the symbol in such a way that it becomes more difficult to follow. To this end, the symbol can move faster and faster, or its acceleration can vary with an increasing amplitude. It is also possible to change the movement pattern so that it becomes more difficult to follow the stimuli, typically by decreasing a radius with which the symbol turns.

Once a tested person has lost track of the stimulus as detected with the eyetracking unit 9, measures may be taken so that the tested person again discovers the symbol with reversed or reset stimulus parameters. For instance, the symbol can have a designated starting position marked on the screen where it reappears after being lost. Also, an additional indicator may appear on the screen, e.g. an arrow temporarily pointing at the symbol or a larger ring encircling the symbol. The symbol may also begin to flash, etc. In general, an indicator is provided at the symbol to allow the tested person to regain contact. By such means the tested person regains view of the stimulus, and the test can be repeated to verify the result, or alternatively a different test can be performed. As an alternative, the change of the stimulus parameter may be reversed when the tested person loses of visual contact with the symbol.

Not only acuity testing can be performed.

### Contrast Testing

In addition to the above described acuity testing, specific testing of the eye's capability of distinguishing a pattern with a given contrast can be carried out. This can be done, for instance, by carrying out the testing based on a symbol shrinking in size as shown in FIG. 2, or a test where the pixels of the symbol become increasingly shuffled as in FIG. 3. This may be done until the tested person loses track of the symbol. Then, the test is repeated with a decreased contrast within the symbol and/or with regard to the background, similar for instance to the symbol in FIG. 4. By repeating the test a number of times with different levels of contrast (increasing or decreasing), a more detailed metric of the eyesight capability of the tested person can be achieved.

### Color Vision

It is also possible to include colored features of symbols to simultaneously or sequentially add a colorvision testing capability, for instance by using red and green pixels instead of black and white in the example of FIG. 3. It is possible to vary the colours of a moving symbol or to change the colour of the symbol in between subsequent tests.

### Smooth Pursuit, 3D Testing

It should be understood that the above-described acuity, contrast and color vision testing methods are not useful with all persons to be tested. Some neurological conditions, typically a stroke or a severe concussion may cause that the person to be tested does not meet some basic requirements for following a stimuli on a screen which means that the result will not be correct. Therefore, it may be useful to begin testing with a basic smooth pursuit test. This may be done by performing a basic test where a stimuli moves over the screen in an unpredictable fashion without altering the stimuli, much like following the flight of a fly. The analyzing unit determines whether or not the tested person is able to follow the stimulus by means of the eyetracking function. Typically, this testing may be carried out with the both the tested person's eyes at the same time to optionally also test vergence capability, i.e. the tested person's ability to move the eyes in opposite directions. The testing may be carried out with a three-dimensional movement pattern. This may be done for instance in a setup as shown in FIG. 1B where the tested person's left and right eye can each have a display, which show slightly different images to provide a three-dimensional effect.

If the tested person is unable to perform basic smooth pursuit, carrying out the aforementioned acuity testing or the refractive testing to be described may be more or less meaningless, and the system may output this result. The person may then instead be tested manually, for instance with a traditional Snellen-chart.

This test by itself also provides a neurological assessment which in itself may be useful, for instance in a telemedicine system. By changing the velocity or acceleration of a symbol until the tested person loses track thereof, different metrics related to neurological status can be achieved.

### Refraction Testing

A second use of the general concept is refractive testing, i.e. determining spherical and cylindrical refractive errors for the tested person's eyes. This may be done separately or in combination with acuity testing. Generally, the dependence on optical distance to the stimulus when the tested person looses track of the stimulus is determined.

A very basic example is schematically illustrated in FIG. 5. This example corresponds to the basic setup in FIG. 1. As long as the display unit screen 7 is not spherical with the tested person's eye 19 in the centre of the sphere, the eye-to-screen distance will vary as a symbol or other stimuli moves over the screen 7. In the illustrated example, a symbol 13 displayed straight in front of the tested person's eye 19 will be displayed at a relatively short distance Ds while the symbol 13 when laterally displaced will be shown at a comparatively longer distance Dl. Depending on the tested person's refractive errors, or absence thereof, the ability to follow a displayed symbol may vary depending on the distance to the display. Therefore, a setup as illustrated in FIGS. 1 and 5 may give information about the tested person's refractive errors, spherical and/or cylindrical. For instance if an acuity test is carried out as illustrated in FIG. 2, the distance at which the tested person looses track of the symbol gives additional information about the tested person's eyesight, and repeating the test where other symbol are the same at a different distance may distinguish a refractive error.

It is possible to additionally vary the eye-to-screen distance in other ways that are less dependent on the tested person's gaze angle. For instance, in the basic setup illustrated in FIG. 1, it would be possible to mechanically vary the distance between the user 5 and the screen 7. This can be done either by moving the screen 7 or the headrest 3 or the like that the tested person is connected with.

It is also possible, as illustrated in FIG. 6 to increase the available distance differences by turning the screen 7 about an axis of its plane.

Additionally, as illustrated in FIG. 7, it would be possible to carry out this test using a phoropter 21 as is common with Snellen-charts. The setting used with the phoropter may be determined with initial tests of the above-described type, assessing refractive error by detecting when the tested person is unable to follow the symbol. It is also possible to carry out an automatic initial autorefraction measuring, which is known per se, to determine the initial setting. Eye tracking can be made through a phoropter, preferably a phoropter setting is inputted to the eye tracking device 9 such that its algorithm can be adapted thereto. The phoropter 21 may be manually or automatically controlled. The phoropter could also be any controllable lens unit that allows to control the lens in close proximity to the eye.

Yet another alternative is to use multiple screens 7 on different distances as illustrated in FIG. 8. The screens 7 in the front are located in different distances to the patient an may even be partly transparent. By assessing the respective properties of the stimulus where the tested person looses track of the stimulus at different distances, an estimation of the tested person's refractive error can be made.

### Microperimetry Measurements

The present disclosure may also be relevant for microperimetry, where a sensitivity of different areas of the tested person's fundus is tested. This is typically done to test for age-related macula degeneration, AMD (or ARMD) or any other pathology that includes defects in the central vision of a patient. FIG. 7 illustrates a movement pattern for a stimulus useful to this end.

Early AMD is often characterised by weak or blind sectors in the macula around the fovea in the retina. The present disclosure provides a reliable method of detecting such weak or blind sectors.

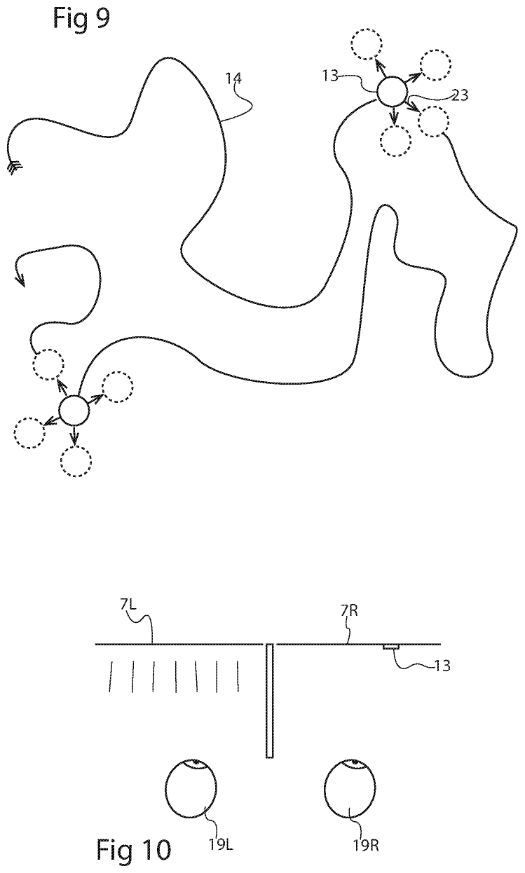

In FIG. 9 stimulus is provided by moving a symbol 13 over a screen along a path 14. The symbol 13, that may have different shapes, moves slowly along the path 14, but at some locations, the symbol makes a quick jump 23 in a direction. This jump may be made by deleting the symbol 23 in at the first location, and displaying the symbol again at the new location. A movement where the symbol is displayed in between those location is not needed. Typically, the jump is made in a direction that deviates from the direction the symbol 13 moved prior to the jump 23. If the macula sector corresponding to the location to which the symbol 13 is moved has deteriorated, it is much likely that the tested person loses track of the symbol. This process may be repeated a number of times where the jump is done in different angles where angle and jump distance makes up stimulus parameters. In this way, different segments of the macula can be tested. Typically, a movement pattern which tests all angular segments a few times can be carried out with a predetermined movement pattern in order to provide a verified result.

### Dual Screens

In the case with a VR headset as shown in FIG. 1B but potentially also in a more stationary basic arrangement as shown in FIG. 1A, it is possible to use different screens for the tested person's right and left eye, respectively. This provides several additional possibilities to accomplish refined test data.

To start with, it is possible to test the right and left eyes in a seamless sequence without the need to sequentially cover the right and left eyes. It is possible still to receive different results from the left and right eye, respectively. For instance, the same moving stimulus may be initially presented to the left and right eyes, but stimulus parameters may vary differently which can accomplish eye metrics related to only one of the eyes.

Secondly, depth may be added to the presented stimulus, and data corresponding to the eyes ability to cooperate may be produced.

Thirdly, one screen, which need not produce any stimuli, e.g. to the left eye, can be used to manipulate pupil size also for the right eye. In this way it is therefore possible to repeat a measurement on the right eye with different pupil sizes without altering the stimulus presented to the right eye. This is illustrated in FIG. 10. In this case there is provided a left display unit 7L in the form of a screen or display which is visible to the left eye 19L, and in the same way a right display unit 7R which is visible to the right eye 19R. The right screen 7R display a stimulus 13 which the right eye 19R attempts to follow. No corresponding stimulus is displayed to the left eye 19L, but that eye is open and usually will follow the right eye's movement. The left screen 7L is instead used to manipulate pupil size. In the illustrated case, the left screen 7L provides a bright light which causes the left eye 19L pupil to contract. With the neurological constitution of the human vision the eyes function in parallel such that the right eye 19R contracts in the same way which affects the test of the right eye. A smaller pupil means a reduced light flow but also a sharper eyesight due to more parallel light. It is therefore possible for instance to repeat a test of the right eye several times under identical conditions but with different pupil sizes to gain improved knowledge of the function of the eye.

In general with the above tests, the analyzing unit can output stimulus metric of the stimulus at the instant when the tested person loses track of the stimulus, e.g. size, contrast, speed for instance. It is however possible also to provide a more elaborated analysis based on such metrics. As indicated in FIG. 1A, a database 12 may contain, for instance, test results of corresponding tests carried out on tested persons whose eyesight capabilities have been assessed in known, more complex tests. For instance, the database may contain information regarding several individuals who have been tested for example with the acuity test described above but also for instance with a traditional manual testing using Snellen charts. Then, the system can readily map a given metric when a tested person loses track of a symbol, for instance, with an acuity metric as produced by a slower and less cost effective method.

The present disclosure is not restricted to the above disclosed examples, and may be varied and altered in different ways within the scope of the appended claims.

1. A device for providing an eye metric, comprising a display unit, producing a visual stimulus to an eye, an eye-tracking unit, measuring the eye's movements in response to said stimulus, and an analyzing unit, outputting a metric result, characterized by
the display unit being configured to produce a moving stimulus with at least one varying stimulus parameter,
the eye-tracking unit being configured to detect the eye loosing visual contact with the stimulus, and
the analyzing unit being configured to provide a metric result based on the value of said stimulus parameter at the time when loss of visual contact was detected.

2. The device according to claim 1, wherein a varying stimulus parameter is the contrast between different parts of a moving symbol and/or between the moving symbol and the background.

3. The device according to claim 2, wherein the symbol comprises a lighter field and a darker field.

4. The device according to claim 3, wherein the contrast between the darker field and the lighter field is gradually decreased.

5. The device according to claim 3, wherein pixels of the darker and the lighter field are increasingly shuffled.

6. The device according to claim 2, wherein the average brightness of the symbol is the same as the background.

7. The device according to claim 1, wherein the stimulus parameter is changed by decreasing the size of a moving symbol.

8. The device according to claim 1, wherein the stimulus parameter is changed by changing the velocity, acceleration or turning radius of a moving symbol.

9. The device according to claim 1, wherein the stimulus is a symbol moving along a path and repeatedly making jumps in different angles.

10. The device according to claim 9, wherein the jump is made in a direction deviating from the symbol's direction of movement prior to the jump.

11. The device according to claim 9, wherein when the tested person loses of visual contact with the symbol, an indicator is provided at the symbol to allow the tested person to regain contact.

12. The device according to any of claim 1, wherein change of the stimulus parameter is reversed when the tested person loses of visual contact with the symbol.

13. The device according to claim 1, wherein the stimulus is provided with different colours.

14. The device according to claim 1, wherein the device includes a positioning unit to keep the tested person at a fixed location, the stimulus includes a moving symbol, and the display unit is configured to produce the moving object at different optical distances from the eye.

15. The device according to claim 14, wherein the display unit is angled with respect to the location of the tested person.

16. The device according to claim 14, wherein the display unit comprises multiple displays at different distances to the location of the eye.

17. The device according to claim 1, wherein the display unit is configured to provide the stimulus in a pattern that is unpredictable to the tested person.

18. The device according to claim 1, wherein the analyzing unit is configured to provide a metric result based on the value of said stimulus parameter at the time when loss of visual contact was detected and based on a database containing data of a plurality of tested persons having carried out a corresponding test.

19. The device according to claim 1, wherein a controllable lens is placed in close proximity to the tested eye.

20. The device according to claim 19, being configured to change cylindrical and spherical values of that lens.

21. The device according to claim 19, where the analyzing unit controls both the lens and the screen.
